(19) United States
(12) Patent Application Publication
(10) Pub. No.: US 2008/0188673 A1
(43) Pub. Date: Aug. 7, 2008

(54) Ether Alcohol-Based Surfactants Having a Reduced Surface Tension and Use Thereof

(75) Inventors: Kathrin Lehmann, Leverkusen (DE); Petra Hinrichs, Bochum (DE); Philipp Tomuschat, Essen (DE); Isabella Ulrich-Brehm, Ratingen (DE)

Correspondence Address: FROMMER LAWRENCE & HAUG, 745 FIFTH AVENUE- 10TH FL., NEW YORK, NY, 10151, US

(73) Assignee: Goldschmidt GmbH, Essen (DE)

(21) Appl. No.: 11/909,179

(22) PCT Filed: Mar. 1, 2006
(86) PCT No.: PCT/EP2006/001853

§ 371 Date: Sep. 20, 2007

(30) Foreign Application Priority Data
Mar. 24, 2005 (EP) 05006514.3

(51) Int. Cl. C07D 303/04; C07C 31/18

(52) U.S. Cl. 549/512; 568/852

(57) ABSTRACT

The invention provides low surface tension surfactants based on ether alcohol and provides for their use as surfactants in aqueous coating formulations, said surfactants being preparable by reacting at least one hydroxy compound of formula (I):

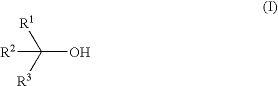

with at least one epoxide of formula (II):

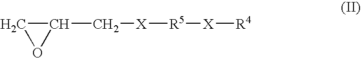

and/or at least one epoxide of formula (V):

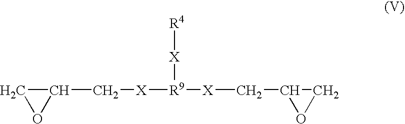

in substantially equivalent amounts of hydroxyl and epoxide groups.

The invention relates to low surface tension surfactants based on ether alcohol and to their use as surfactants in aqueous coating formulations.

Water-based paints and coatings are used on a large scale industrially. Critical to effective wetting of the substrate is the lowering of the surface tension of the aqueous system by means of a surfactant. It is not only the lowering of the static surface tension to a small value that is decisive here, but also the corresponding lowering of the dynamic surface tension. A low dynamic surface tension is needed in particular for high-speed applications: for example, when applying coatings by spraying, or in printing operations. Furthermore, the surfactants used must not disrupt the development of a uniform film, must not cause any turbidity, and should be low-foaming—that is, should not promote the build-up of large amounts of foam.

Although nonionic surfactants such as alkylaryl ethoxylates or alcohol ethoxylates or ethylene oxide (EO)-propylene oxide (PO) copolymers are capable of reducing the static surface tension, the high molecular weight and resultant low molecular mobility of these classes of compounds mean that it is not possible to lower the dynamic surface tension to a value which is acceptable to the user.

Conversely, some anionic surfactants, such as the sodium salts of monoalkyl or dialkyl sulfosuccinates, are able effectively to reduce the dynamic surface tension, but using them leads to severe build-up of foam in application, and the finished coating reacts sensitively to water.

More recently a new class of surfactants has been developed based on acetylenic glycols and their alkoxylates. The properties of these surfactants are situated between those of the surfactants outlined above. With these new surfactants it is possible to reduce both the static and the dynamic surface tension, with the values which can be achieved not entirely matching those of the nonionic and anionic surfactants. But, on the plus side, these surfactants provide comparatively low-foam formulations (EP-B-0 897 744, U.S. Pat. No. 2,997,447).

In view of these properties, surfactants of this kind have been able to establish themselves convincingly in numerous applications. Their properties are primarily attributed to the rigid acetylenic alkyl spacer, which, as a result of the restricted degrees of freedom, dictates a kind of preorientation of polar and nonpolar groups. Responsibility for these properties is additionally ascribed to the small distance between the polar groups and to the low molecular weight (<300 g/mol), which allows the surfactant molecules to be highly mobile.

A problem with compounds of this type is that, in applications, foam build-up reoccurs after a very short time. For the user, on the other hand, it is very important to prevent this new foam build-up for as long as possible. The alternative would be to add defoamers, whose possible consequences include unwanted disruptions to the development of the coating film and problems with interlayer adhesion.

Furthermore, the ecotoxicological evaluation of products based on 2,4,6,8-tetramethyl-5-decynediol is not unproblematic, and the products, additionally, are labeled at least “Xi” (irritant). Either only solid products are available to the paint manufacturer from this class of substance, or the substance is supplied for ease of handling as a 50% strength solution in various solvents, such as ethylene glycol (classified “Xn” (harmful), suspected of having reproductivity effects). Alkoxylates of these substances, although likewise effective, display a much lower potential for foam reduction.

Products which find use as surfactants in low-viscosity aqueous or solventborne paints, inks and other coating materials ought preferably to be neat liquids.

It is therefore clear that the need for environment-friendly surfactants which can be given a ecotoxico-logical evaluation, particularly for aqueous coating systems, has not yet been structurally solved with regard to foam prevention and foam inhibition. Optimizing individual properties is possible, but is achieved generally at the expense of the other required parameters,

There was therefore a need to enhance the overall profile of properties and to provide compounds which not only allow effective reduction in static and dynamic surface tension but also prevent foam build-up/new foam build-up effectively for a long time.

In an effort to overcome the disadvantages of the prior art and to provide compounds which significantly reduce both static and dynamic surface tension and at the same time effectively inhibit the (re)formation of foam for a long time it has now surprisingly been found that this objective can be achieved by means of ether alcohols preparable by reacting compounds containing a hydroxyl group with glycidyl compounds.

The invention accordingly first provides ether alcohols obtained by reacting one or more hydroxyl compounds of formula (I)

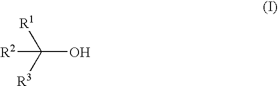

in which
R¹ can be a branched or unbranched, aromatic or nonaromatic, saturated or unsaturated residue with or without heteroatom substituents and containing 1 to 9 carbon atoms or can be R^{1a}, with the meaning as follows:
R^{1a} is oxyalkylene residue of the formula (III)

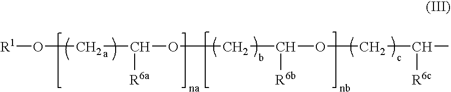

where
R^{6a},R^{6b} and R^{6c} independently of one another are hydrogen, or other, branched or unbranched, unsaturated or saturated residues with or without heteroatom substituents and containing 1 to 10 carbon atoms, preferably 1 to 5, correspond optionally to aromatic residue having 6 to 8 carbon atoms, and in particular are methyl and/or ethyl residues, and
a, b, and c independently of one another are numbers between 0 to 10, preferably 1 to 3, and
na and nb independently of one another are numbers between 0 and 25, with 1<na+nb<25, preferably with 1<na+nb<20, more particularly with 1<na+nb<12, with the proviso that both a random and a blockwise arrangement of the oxyalkylene units may be present,
R² and R³ independently of one another can be hydrogen or one of the residues R¹,
   with at least one epoxide of the formula (II)

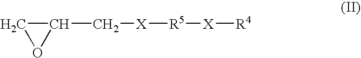

where
X is an oxygen or a carboxyl group,
R⁴ is either also 2,3-epoxypropyl or R^{1a} or any other, branched or unbranched, saturated or unsaturated residue with or without heteroatom substituents,
   with the proviso that there is on average more than one, preferably more than one and a half, 2,3-epoxypropyl residues in the molecule,
R⁵ is either a branched or unbranched, saturated or unsaturated, aromatic or nonaromatic residue with or without heteroatom substituents and containing 1 to 30, preferably 2 to 20, carbon atoms, more preferably containing 3 to 8 carbon atoms, or
R⁵ is an oxyalkylene residue R^{1b} of the formula (IIIb)

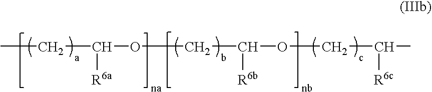

in which a, b, c, na, nb, R^{6a}, R^{6b} and R^{6c} are as defined above or
R⁵ is a residue of the formula (IV)

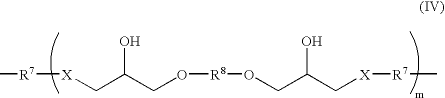

where
R⁷ is a branched or unbranched, saturated or unsaturated, aromatic or nonaromatic residue with or without heteroatom substituents and containing 1 to 30, preferably 2 to 20, carbon atoms, more preferably containing 3 to 8 carbon atoms, or is an oxyalkylene residue R^{1b} of the formula (IIIb), and
R⁸ is a branched or unbranched, saturated or unsaturated, aromatic or nonaromatic residue with or without heteroatom substituents and containing 1 to 30, preferably 2 to 20, carbon atoms, more preferably containing 3 to 8 carbon atoms, or likewise is an oxyalkylene residue R^{1b} of the formula (IIIb), and
m is 1 or 2.

The invention further provides ether alcohols obtained by reacting one or more alcohols of formula (I) above with at least one epoxide of formula (V)

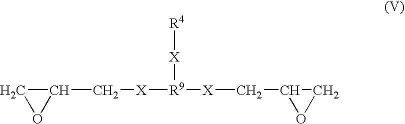

where
X and R⁴ are as already defined for formula (II), with the proviso that there are on average more than two 2,3-epoxypropyl residues in the molecule, and
R⁹ can be a branched or unbranched, saturated or unsaturated residue with or without heteroatom substituents or the residue R^{1b} of the formula (IIIb), in which the residues R^{6a}, R^{6b} and R^{6c} independently of one another are as defined above but at least once are branched or unbranched, saturated or unsaturated alkylene residues with or without heteroatom substituents and containing 1 to 10 carbon atoms, preferably 1 to 5, more particularly are methylene and/or ethylene residues.

The invention further provides ether alcohols of the general formulae (VI) and (VII)

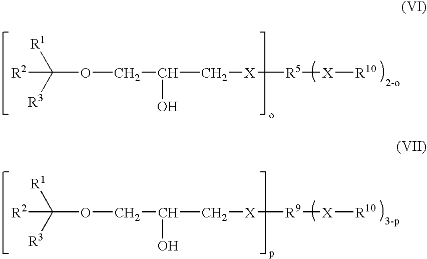

in which
X is an oxygen or a carboxyl group,
R¹ can be a branched or unbranched, aromatic or nonaromatic, saturated or unsaturated residue with or without heteroatom substituents and containing 1 to 9 carbon atoms or can be R^{1a}, with the meaning as follows:
R^{1a} is oxyalkylene residue of the formula (III)

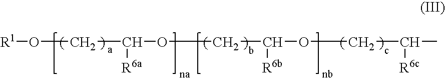

where
R^{6a},R^{6b} and R^{6c} independently of one another are hydrogen, or other, branched or unbranched, unsaturated or saturated residues with or without heteroatom substituents and containing 1 to 10 carbon atoms, preferably 1 to 5, correspond optionally to aromatic residue having 6 to 8 carbon atoms, and in particular are methyl and/or ethyl residues, and
a, b, and c independently of one another are numbers between 0 to 10, preferably 1 to 3, and
na and nb independently of one another are numbers between 0 and 25, with 1<na+nb<25, preferably with 1<na+nb<20, more particularly with 1<na+nb<12, with the proviso that both a random and a blockwise arrangement of the oxyalkylene units may be present,
R² and R3 independently of one another can be hydrogen or one of the residues R¹,
R⁵ is either a branched or unbranched, saturated or unsaturated, aromatic or nonaromatic residue with or without heteroatom substituents and containing 1 to 30, preferably 2 to 20, carbon atoms, more preferably containing 3 to 8 carbon atoms, or R⁵ is an oxyalkylene residue R^{1b} of the formula (IIIb)

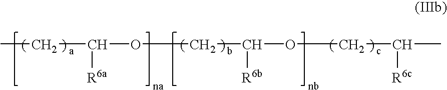

in which a, b, c, na, nb, R^{1a}, R^{6b} and R^{6c} are as defined above, or R⁵ is a residue of the formula (IV)

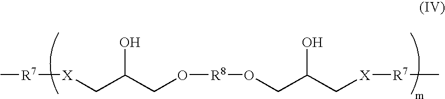

where
R⁷ is a branched or unbranched, saturated or unsaturated, aromatic or nonaromatic residue with or without heteroatom substituents and containing 1 to 30, preferably 2 to 20, carbon atoms, more preferably containing 3 to 8 carbon atoms, or is an oxyalkylene residue R^{1b} of the formula (IIIb), and
R⁸ is a branched or unbranched, saturated or unsaturated, aromatic or nonaromatic residue with or without heteroatom substituents and containing 1 to 30, preferably 2 to 20, carbon atoms, more preferably containing 3 to 8 carbon atoms, or likewise is an oxyalkylene residue R^{1b} of the formula (IIIb), and
R⁹ can be a branched or unbranched, saturated or unsaturated residue with or without heteroatom substituents or the residue R^{1b} of the formula (IIIb), in which the residues R^{6a}, R^{6b} and R^{6c} independently of one another are as defined above but at least once are branched or unbranched, saturated or unsaturated alkylene residues with or without heteroatom substituents and containing 1 to 10 carbon atoms, preferably 1 to 5, more particularly are methylene and/or ethylene residues,
R¹⁰ residues are any desired residues from the group of branched or unbranched, saturated or unsaturated residues with or without heteroatom substituents,
o is 1 to 2, preferably >1.5 to 2, and in particular about 2, and
p is 2 to 3, preferably >2.5 to 3, and in particular about 3.

The invention additionally provides for the use of the ether alcohols of the invention as additives in aqueous formulations, especially aqueous formulations for surface coatings, paints, printing inks or varnishes.

The invention further provides aqueous formulations comprising at least one of the ether alcohols of the invention, such wetting agents being used normally in amounts from 0.05% to 5%, preferably from 0.1% to 3%.

The alcohols and glycidyl ethers/esters used in accordance with the invention are industrial products which can be employed in the form of their respective commercially customary specifications, although in specialty applications of the ether alcohols of the invention higher levels of purity may be required.

A particularly preferred residue R¹ in the alcohol is the n-propyl, isopropyl, n-butyl, isobutyl, 2-butyl, isononyl residue, the residue R^{1a}.

Diglycidyl ethers used are preferably ethylene glycol diglycidyl ether, 1,2-propanediol diglycidyl ether, 1,3-propanediol diglycidyl ether, 1,3-butanediol diglycidyl ether, 1,4-butanediol diglycidyl ether, 1,6-hexanediol diglycidyl ether, neopentyl glycol diglycidyl ether, cyclohexanedimethanol diglycidyl ether, diethylene glycol diglycidyl ether, dipropylene glycol diglycidyl ether, resorcinol diglycidyl ether, 2,2-bis[4-(glycidyloxy)phenyl]propane, bis(4-glycidyl-oxyphenyl)methane or bisphenol A propoxylate (1-PO/phenol)diglycidyl ether.

Particular preference is given to using diglycidyl ethers of polyalkylene glycols, such as polyethylene glycol diglycidyl ether, polypropylene glycol diglycidyl ether, polybutylene glycol diglycidyl ether, and diglycidyl ethers of other polyoxyalkylene compounds, which can be homopolymers or copolymers with a blockwise or random construction, whose alkylene groups optionally are branched or carry aromatic residues and whose average molecular weight is up to 1500 g/mol, more preferably between 200 and 1000 g/mol.

As triglycidyl ethers it is preferred to use glycerol triglycidyl ether, trimethylolpropane triglycidyl ether, and triphenylolmethane triglycidyl ether.

As diglycidyl and triglycidyl esters it is possible to use all corresponding esterified dicarboxylic or tricarboxylic acids of aliphatic, branched, cyclo-aliphatic, aromatic or aromatic-aliphatic structure, preference being given to employing diglycidyl malonate, diglycidyl succinate, diglycidyl glutarate, diglycidyl adipate, diglycidyl 1,2-cyclohexane-dicarboxylate, and diglycidyl terephthalate.

Particularly preferred glycidyl compounds are those having two or three functional groups, such as 1,4-butanediol diglycidyl ether, 1,6-hexanediol diglycidyl ether, neopentyl glycol diglycidyl ether, trimethylolpropane triglycidyl ether, diglycidyl 1,2-cyclohexanedicarboxylate, polyethylene glycol diglycidyl ether, polypropylene glycol diglycidyl ether, and poly(ethylene-stat./block-propylene glycol) diglycidyl ether.

Glycidyl compounds of the above-defined formula (II)

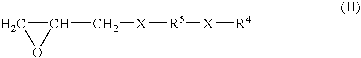

in which the residue R⁵ corresponds to the above-described formula (IV)

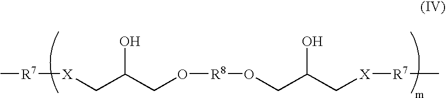

are obtained by reacting a diol with a commercially customary diglycidyl compound by processes which are known per se, the diglycidyl compound being used in an at least 4-fold access.

For the preparation of the ether alcohols claimed, alcohols and glycidyl compounds are preferably used in approximately equivalent amounts based on reactive hydroxyl and epoxide groups. The basis for calculation are the OH number and epoxide values which are familiar to the skilled worker.

Experimental Section:

Complete conversion in all reactions was verified by ¹H NMR measurements.

### EXAMPLE 1

### Reaction of 1,4-butanediol diglycidyl ether with diisobutylcarbinol

27 g (0.19 mol) of diisobutylcarbinol and 0.1 g (0.2% by weight) of BF₃-acetic acid are heated to 90° C. under nitrogen with stirring. Subsequently 20 g (0.09 mol) of 1,4-butanediol diglycidyl ether (epoxy value: 14.8%) are slowly added dropwise. At the end of the addition the mixture is stirred at 110° C. for 4 hours. After the end of reaction the mixture is cooled to give a clear, colorless liquid.

### EXAMPLE 2

### Reaction of 1,4-butanediol diglycidyl ether with isobutanol

20 g (0.27 mol) of isobutanol and 0.05 g (0.1% by weight) of BF₃-acetic acid are heated to 50° C. under nitrogen with stirring. Subsequently 29.2 g (0.14 mol) of 1,4-butanediol diglycidyl ether (epoxy value: 14.8%) are slowly added dropwise. At the end of the addition the mixture is stirred at 90° C. for 4 hours. After the end of reaction the mixture is cooled to give a clear, colorless liquid.

### EXAMPLE 3

### Reaction of 1,4-butanediol diglycidyl ether with 2-butanol

20 g (0.27 mol) of 2-butanol and 0.05 g (0.1% by weight) of BF₃-acetic acid are heated to 50° C. under nitrogen with stirring. Subsequently 29.2 g (0.14 mol) of 1,4-butanediol diglycidyl ether (epoxy value: 14.8%) are slowly added dropwise. At the end of the addition the mixture is stirred at 90° C. for 3 hours. After the end of reaction the mixture is cooled to give a clear, colorless liquid.

### EXAMPLE 4

### Reaction of 1,4-butanediol diglycidyl ether with 2-ethyl-1-butanol

20 g (0.2 mol) of 2-ethyl-1-butanol and 0.04 g (0.1% by weight) of BF₃-acetic acid are heated to 50° C. under nitrogen with stirring. Subsequently 21.2 g (0.1 mol) of 1,4-butanediol diglycidyl ether (epoxy value: 14.8%) are slowly added dropwise. At the end of the addition the mixture is stirred at 90° C. for 4 hours. After the end of reaction the mixture is cooled to give a clear, colorless liquid.

### EXAMPLE 5

### Reaction of 1,4-butanediol diglycidyl ether with 2,2-dimethyl-1-propanol

20 g (0.2 mol) of 2,2-dimethyl-1-propanol and 0.05 g (0.1% by weight) of BF₃-acetic acid are heated to 50° C. under nitrogen with stirring. Subsequently 25.4 g (0.1 mol) of 1,4-butanediol diglycidyl ether (epoxy value: 14.3%) are slowly added dropwise. At the end of the addition the mixture is stirred at 90° C. for 3 hours. After the end of reaction the mixture is cooled to give a clear, pale yellow liquid.

### EXAMPLE 6

### Reaction of neopentyl glycol diglycidyl ether with 2-butanol

27.4 g (0.37 mol) of 2-butanol are heated to 50° C. under nitrogen with stirring. Subsequently 0.16 g (0.2% by weight) of BF₃-acetic acid are added and 50 g (0.18 mol) of neopentyl glycol diglycidyl ether (epoxy value: 10.5%) are slowly added dropwise. At the end of the addition the mixture is stirred at 90° C. for 2 hours. After the end of reaction the mixture is cooled to give a clear, colorless liquid.

### EXAMPLE 7

### Reaction of 1,6-hexanediol diglycidyl ether with 2-butanol

30.2 g (0.41 mol) of 2-butanol and 0.17 g (0.2% by weight) of BF₃-acetic acid are heated to 50° C. under nitrogen with stirring. Subsequently 57.1 g (0.2 mol) of 1,6-hexanediol diglycidyl ether (epoxy value: 11.4%) are slowly added dropwise. At the end of the addition the mixture is stirred at 90° C. for 3.5 hours. After the end of reaction the mixture is cooled to give a clear, colorless liquid.

### EXAMPLE 8

### Reaction of polypropylene glycol diglycidyl ether with 2-butanol

27.4 g (0.37 mol) of 2-butanol are heated to 50° C. under nitrogen with stirring. Subsequently 0.16 g (0.2% by weight) of BF₃-acetic acid are added and 50 g (0.18 mol) of polypropylene glycol diglycidyl ether (average molar mass: 270 g/mol, epoxy value: 11.8%) are slowly added dropwise. At the end of the addition the mixture is stirred at 90° C. for 1.5 hours. After the end of reaction the mixture is cooled to give a clear, colorless liquid.

### EXAMPLE 9

### Reaction of trimethylolpropane triglycidyl ether with isobutanol

15.4 g (0.21 mol) of isobutanol and 0.09 g (0.2% by weight) of BF₃-acetic acid are heated to 50° C. under nitrogen with stirring. Subsequently 30 g (0.07 mol) of trimethylolpropane triglycidyl ether (epoxy value: 11.1%) are slowly added dropwise. At the end of the addition the mixture is stirred at 90° C. for 3.5 hours.

After the end of reaction the mixture is cooled to give a clear, colorless liquid.

### EXAMPLE 10

### Reaction of polypropylene glycol diglycidyl ether with isobutanol

27.4 g (0.37 mol) of isobutanol are heated to 50° C. under nitrogen with stirring. Subsequently 0.16 g (0.2% by weight) of BF₃-acetic acid are added and 50 g (0.18 mol) of polypropylene glycol diglycidyl ether (average molar mass: 270 g/mol, epoxy value: 11.8%) are slowly added dropwise. At the end of the addition the mixture is stirred at 90° C. for 1.5 hours. After the end of reaction the mixture is cooled to give a clear, colorless liquid.

### EXAMPLE 11

### Reaction of diglycidyl cyclohexane-1,2-dicarboxylate with 2-butanol

29.5 g (0.4 mol) of 2-butanol and 0.17 g (0.2% by weight) of BF₃-acetic acid are heated to 50° C. under nitrogen with stirring. Subsequently 56.8 g (0.2 mol) of diglycidyl cyclohexane-1,2-dicarboxylate are slowly added dropwise. At the end of the addition the mixture is stirred at 90° C. for 4.5 hours. After the end of reaction the mixture is cooled to give a clear, colorless liquid.

### EXAMPLE 12

### Reaction of hexanediol diglycidyl ether with alkoxylated isononanol

28.7 g (0.06 mol) of alkoxylated isononanol containing 4 mol of ethylene oxide units and 2 mol of propylene oxide units per mole are heated to 50° C. under nitrogen with stirring. Subsequently 0.07 g (0.2% by weight) of BF₃-acetic acid are added and 8.6 g (0.03 mol) of 1,6-hexanediol diglycidyl ether (epoxy value: 11.4%) are slowly added dropwise. At the end of the addition the mixture is stirred at 90° C. for 3 hours. After the end of reaction the mixture is cooled to give a clear, colorless liquid.

### EXAMPLE 13

### Reaction of hexanediol diglycidyl ether with alkoxylated 2-ethyl-1-hexanol

The reaction was carried out in analogy with example 12 in the same molar quantities.

Application Tests:

For the testing of new wetting agents a skilled worker performs a series of overview tests in order to assess not only the inhibitory and/or preventative effect on foam but also the rapid, surfactant-initiated destruction of foam formed in a system by other surface-active substances. Another important criterion for grading surfactants is their long-term effect in the sense of preventing foam even after storage of the corresponding system equipped with the wetting agent. This matter is one of particular importance, since foam prevention during coating-material production is fundamentally different from foam-free application by means of spraying, knifecoating, pouring, etc; further addition of the surfactant during application is undesirable.

Dynamic Surface Tension:

Determining the dynamic surface tension of the formulated systems is essential to be able to estimate the rate at which a wetting agent molecule reaches a newly generated interface in order to be able to make an active contribution to destroying foam.

These values are determined using the online tensiometer t 60 from SITA Messtechnik GmbH. This instrument measures the dynamic surface tension in accordance with the principle of maximum bubble pressure: the internal force of attraction of a liquid also compresses those air bubbles present in the liquid. The resultant pressure increases as the bubble radius falls. It is this pressure, increased in relation to the ambient pressure, that is utilized for the bubble pressure method. A gas stream is passed through a capillary, which is dipped in a liquid. The bubble surface which forms becomes curved and continuously reduces the radius of the bubble. The pressure increases up to a maximum value. At this value the bubble has attained its smallest radius, the capillary radius, and forms a hemisphere. When this point is exceeded the bubble bursts and tears away from the capillary, allowing a new bubble to form. This produces a characteristic pressure curve, which is evaluated in order to determine the surface tension. In other words, the smaller the value in the case of low bubble frequency, the more effective the wetting agent in wetting a low-energy surface. The smaller the difference between the value at low bubble frequency and the value at high bubble frequency, the more capable the wetting agent of orienting itself to newly created surfaces—that is, in being effective even during highly dynamic application operations.

The wetting agents claimed in accordance with the invention were evaluated by carrying out the tests set out in greater detail below.

Foam Inhibition Effect:

A defined amount of wetting agent is added to a defined amount of a test system and is incorporated using a toothed-wheel disk at 1500 rpm for 1 minute. Subsequently air is introduced at 3000 rpm for 1 minute, and foam produced. The resulting foam height is read off and viewed in comparison with the foam height reached in the absence of the wetting agent. Thereafter a measurement is made of the time taken for the foam to go down completely, something which generally does not happen at all in the absence of wetting agents.

Assessment of Foam Build-Up and of Spontaneous Defoaming:

Foam is built up in a defined amount of a test system using a perforated disk (see below) at 2000 rpm for 1 minute. Then a defined amount of wetting agent is placed on the foam and the occurrence of spontaneous defoaming is assessed visually (bursting air bubbles, “prickling” on the surface) and graded as absent (−), present (±) or very characteristic (+).

Shearing with the perforated disk is then repeated at 2000 rpm for one minute. This time a stopwatch is used to record the time which elapses before foam builds up again. If a wetting agent is able to prevent foam building up again, it is classified, with “>60 s”, as very active.

A defined amount of this sample is subsequently introduced into a measuring cylinder and the foam height is recorded by reporting ml of foam and is compared with a blank sample.

The perforated disk employed actually comprises three disks arranged one above the other on a spindle (disk thickness 3 mm, disk diameter 25 mm) and each having three holes (diameter: 5 mm). The distance between the individual disks is 9 mm and they rotate vertically by 120° on the spindle. This apparatus allows optimum introduction of macrofoam and microfoam, such as occurs in painting application operations (such as rolling or spraying, for example) and production processes and can be prevented by suitable wetting agents.

Long-Term Effect:

Following storage of the twice-sheared sample (see test described above) for 4 to 14 days the sample is again stirred with the perforated disk at 2000 rpm for 1 minute and again the resulting foam height of the sample is read off in a measuring cylinder. Where there is hardly any difference between these values and the original determination, the wetting agent is still available in the system and hence is also found to be stable to hydrolysis.

Viscosity:

Surfactants incorporated into inks, paints and other coating materials frequently give rise to unwanted changes in the viscosity of the system, which then, as a result of thickening effects, lead, for example, to fundamentally different film thicknesses during application, and so jeopardize the economics. It is therefore necessary to evaluate the coating material system with surfactant added in comparison to the unsheared blank sample without additive. For this purpose there are a variety of rheometers available, but the one used here is the RC 20-CPS from Europhysics. The program employed measures from 100 [1/s] to 1000 [1/s] in 180 seconds, using a plate/cone geometry.

Incompatibility:

In transparent systems even slight turbidity is a pointer to incompatibility between the surfactant and the surrounding matrix, which is undesirable. In order to ensure that the foam prevention effect of the surfactant is not bought at the expense of turbid clearcoats or of cratering, the skilled worker applies the coating material in question to different substrates for the purpose of visual evaluation (e.g., black PVC film or transparent PE film).

In the following tests the wetting agents of the invention are labeled S1 to S6.

S1 Example 3

S2 Example 8

S3 Example 10

S4 Example 6

S5 Example 7

S6 Example 9

S7 Example 12

Noninventive, comparative examples are the following wetting agents, which are supplied as commercial products for aqueous systems and can be characterized in accordance with the details below.

C1 2,4,7,9-tetramethyl-5-decyne-4,7-diol in ethylene glycol (50% strength solution)

C2 2,4,7,9-tetramethyl-5-decyne-4,7-diol ethoxylate

C3 fatty alcohol alkoxylate with a molar weight of about 500 g/mol

The aforementioned inventive and commercially customary wetting agents are deployed in the standard formulations below.

Water-Based Printing Ink Formulation:

50 g of ink, consisting of:

| | |
|---|---|
| JonCryl ® 8085 (43% ammoniacal solution of | 29.4 g |
| an acrylate resin)¹⁾ | |
| JonCryl ® ECO 2189 (glycol-ether-free, film- | 44.1 g |
| forming polymer dispersion)¹⁾ | |
| JonCryl ® ECO 2177 (glycol-ether-free, film- | 17.7 g |
| forming polymer dispersion)¹⁾ | |
| JonWax ® 35 (polyethylene wax emulsion)¹⁾ | 4.9 g |
| demineralized water | 2.9 g |

¹⁾Johnson Polymer

are weighed out into a 100 ml glass bottle, 0.5% of active matter of wetting agent is stirred in using a 2.5 cm toothed-edge disk at 1500 rpm for 1 minute, and the mixture is then foamed at 3000 rpm for 1 minute. The fill level (solution+foam) is read off in the glass bottle using a ruler and the time taken for the foam to collapse, in minutes, is determined using a stopwatch.

For determining the dynamic surface tension, 12 g of water are added to 48 g of ink containing 0.5% wetting agent. The mixture is homogenized by simple shaking.

TABLE 1
Results in a water-based printing ink:

| | | | Dynamic surface tension |
|---|---|---|---|
| Wetting | Foam | Time to foam | with 2 and 10 bubbles/sec |
| agent | [cm] | collapse [min] | [mN/m] |
| none | 7.0 | stable, >12 h | 39.3-49.4 |
| S1 | 5.3 | 360 | 38.1-42.3 |
| S2 | 5.3 | stable, >12 h | 37.9-42.3 |
| S3 | 5.2 | stable, >12 h | 38.0-42.2 |
| S4 | 4.5 | 240 | 37.6-42.1 |
| S5 | 4.5 | stable, >12 h | 39.7-43.4 |
| S6 | 5.2 | 60 | 37.2-43.9 |
| S7 | 4.8 | 120 | 38.0-42.4 |
| S8 | 4.2 | 60 | 37.2-41.6 |
| C1 | 6.0 | stable, >12 h | 37.6-40.7 |
| C2 | 5.5 | stable, >12 h | 37.4-44.8 |
| C3 | 6.7 | stable, >12 h | 38.3-44.9 |

Table 1 shows that using the wetting agents claimed in accordance with the invention reduces foam build-up as compared with the blank sample and with the comparative examples. With the inventively claimed Examples S4, S6, S7 and S8, indeed, products were created which also guarantee rapid foam collapse. As compared with the blank sample, all investigated Examples S1 to S8 feature the capacity to achieve significant reduction of the dynamic surface tension values as well. The slightly greater reduction in these values for the comparative sample C1 originates not from the surfactant itself but rather from its unavoidably accompanying ethylene glycol [labeled “Xn” (harmful), suspected of having reproductive effects]. The inventively claimed class of the ether alcohol-based surfactants can therefore be evaluated in this printing ink as a low-foam, surface tension-reducing additive group, which in its 100% pure liquid presentation form is easy to incorporate and hence is user-friendly.

Water-Based Automotive Finish I:

50 g of a mixture of 2 parts of aliphatic polyurethane-acrylic hybrid dispersion Daotan® VTW 6264 (Solutia) and 1 part of DI (deionized) water in a vessel (diameter: 65 mm) are foamed at 2000 rpm for 1 minute using a perforated disk (for description see above). 0.2% of active matter of wetting agent ingredient is placed on the resulting foam, and the spontaneous defoaming is observed. This is followed by shearing again at 2000 rpm for 1 minute, after which the time taken for the foam to build up again is measured using a stopwatch. If the foam does not build up again, the evaluation is reported as >60 seconds.

Immediately following the shearing operation, 25 g of this sample are introduced into a 100 ml measuring cylinder, and the fill level is read off in ml.

In order to assess the stability to hydrolysis and the storage stability the sample after four days is again sheared at 2000 rpm for 1 minute and the foam height of 25 g is determined using a 100 ml measuring cylinder.

TABLE 2
Results in an aqueous automotive finish I:

| | | | Foam value | |
|---|---|---|---|---|
| | | Renewed | instantaneous | Foam value |
| | | build-up of | [ml/25 g] | after |
| Wetting | Spontaneous | foam | (Residual | 4 days |
| agent | defoaming* | [sec] | foam) | [ml/25 g] |
| blank | n/a | n/a | 44 | 46 |
| value | | | | |
| S1 | + | >60 | 28 | 30 |
| S2 | + | 60 | 30 | 33 |
| S3 | + | 60 | 30 | 30 |
| S4 | + | >60 | 32 | 33 |
| S5 | + | 45 | 33 | 33 |
| S8 | + | >60 | 27 | 28 |
| C1 | + | 45 | 30 | 32 |
| C2 | +/− | >60 | 37 | 40 |
| C3 | + | 45 | 30 | 33 |

*(−) absent, (+/−) present, (+) very marked

The compounds of the invention exhibit effective spontaneous defoaming. On its own this property of the innovative surfactant is extremely valuable from a performance standpoint for the coatings manufacturer, and is therefore of very great interest. The particular products S1 to S4 and S8, moreover, are notable for particularly low residual foam values, and also allow long-lasting foam prevention in the event of further introduction of shearing. As a consequence it is unnecessary to add additional wetting agent or additional defoamer to the automotive finish system, even after storage.

Essential to the Positive Evaluation of the Compounds of the Invention here is the Summation of the Important Properties in One Structure:

spontaneous defoaming

+ renewed foam build-up absent or very late

+ optimum reduction of foam (instantaneous+ after storage)

Water-based Automotive Finish II:

0.5% of surfactant is added to 10 g of a water-dilutable, self-crosslinking alkyd resin containing urethane groups, Resydrol VAZ 5541 (Solutia), and the surfactant is incorporated by stirring at 3600 rpm using a Hausschild Speedmixer for one minute.

After three days the viscosity of the samples is determined using an RC 20-CPS viscometer from Europhysics at 500 revolutions/second.

In addition the finished material is drawn down onto aluminum at 50 μm using a box-type applicator, for the purpose of an assessment of the compatibility.

TABLE 3
Results of an aqueous automotive finish II

| | Viscosity | Characterization of |
|---|---|---|
| Wetting agent | [mPas] | coating film |
| blank value | 124 | +++ |
| S3 | 125 | +++ |
| S4 | 128 | ++− |
| S5 | 126 | +++ |
| C1 | 138 | +−− |
| C2 | 132 | −−− |
| C3 | 136 | ++− |

(+++) = no defects
(−−−) = severe defects

Table 3, with the corresponding low viscosities, which for the inventively claimed compounds are at the same level as that of the pure automotive finish system without wetting agent (blank value), illustrates that there is virtually no increase in viscosity, in contrast to the comparative examples.

Particularly in the sensitive thin-film automotive finish applications, therefore, a way has been found to reproducible film thickness build-up and surface image. At the same time the effective removal of air from the system becomes clear, as can be demonstrated simply from the viscosities, and additionally through a very good surface image of the finishes, in the form of defect-free films.

Summary:

The compounds claimed in accordance with the invention can be used without reserve as wetting agents in aqueous paints, inks, and other coating materials, since they significantly lower surface tension. As a consequence, in a hitherto unknown profile of properties, they combine spontaneous defoaming properties, which are utilized during coatings preparation and application, with strong foam destruction (residual foam values zero or very low). The latter property is also bound after storage of the coating material, which implies that the compounds claimed in accordance with the invention are not subject to hydrolytic attack—that is, they are surprisingly stable. The compounds claimed in accordance with the invention produce foam destruction not at the expense of a disrupted surface image, and so even sensitive automotive finish surfaces can be applied without disruption.

1. An ether alcohol prepared by reacting at least one hydroxy compound of formula (I) 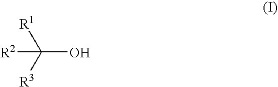
in which
R¹ is a branched or unbranched, aromatic or nonaromatic, saturated or unsaturated residue with or without heteroatom substituents and containing 1 to 9 carbon atoms or is R^{1a}, with the meaning as follows:
R^{1a} is oxyalkylene residue of the formula (III) 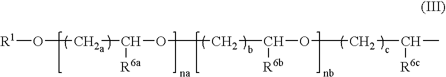
where
R^{6a},R^{6b} and R^{6c} independently of one another are hydrogen, methyl, ethyl, or another, branched or unbranched, unsaturated or saturated residue with or without heteroatom substituents and containing 1 to 10 carbon atoms, correspond optionally to aromatic radical having 6 to 8 carbon atoms, and
a, b, and c independently of one another are numbers between 0 and 3, and
na and nb independently of one another are numbers between 0 and 25, with 1<na+nb<25, with the proviso that both a random and a blockwise arrangement of the oxyalkylene units may be present,
R² and R³ independently of one another can be hydrogen or one of the residues R¹,
with at least one epoxide of the formula (II) 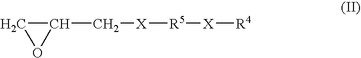
where
X is an oxygen or a carboxyl group,
R⁴ is 2,3-epoxypropyl or a branched or unbranched, saturated or unsaturated residue with or without heteroatom substituents, with the proviso that there is on average more than one, 2,3-epoxypropyl residues in the molecule,
R⁵ is a branched or unbranched, saturated or unsaturated, aromatic or nonaromatic residue with or without heteroatom substituents and containing 1 to 30 carbon atoms, or
R⁵ is an oxyalkylene residue R^{1b} of the formula (IIIb) 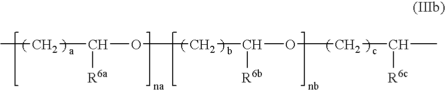
in which a, b, c, na, nb, R^{1a}, R^{6b} and R^{6c} are as defined above
or
R⁵ is a residue of the formula (IV) 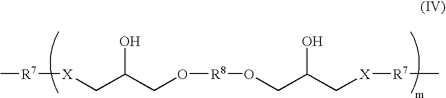
where
R⁷ is a branched or unbranched, saturated or unsaturated, aromatic or nonaromatic residue with or without heteroatom substituents and containing 1 to 30 carbon atoms, or is an oxyalkylene residue R^{1b} of the formula (IIIb), and
R⁸ is a branched or unbranched, saturated or unsaturated, aromatic or nonaromatic residue with or without heteroatom substituents and containing 1 to 30 carbon atoms, or likewise is an oxyalkylene residue R^{1b} of the formula (IIIb), and
m is 1 or 2,
and/or at least one epoxide of formula (V) 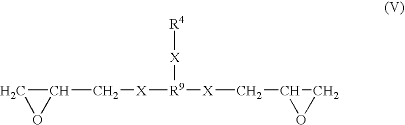
where
X and R⁴ are as already defined for formula (II), with the proviso that there are on average more than two 2,3-epoxypropyl residues in the molecule, and
R⁹ can be a branched or unbranched, saturated or unsaturated residue with or without heteroatom substituents or the residue R^{1b} of the formula (IIIb), in which the residues R^{6a}, R^{6b} and R^{6c} independently of one another are as defined above but at least once are branched or unbranched, saturated or unsaturated alkylene residues with or without heteroatom substituents and containing 1 to 10 carbon atoms.

2. A compound as claimed in claim 1, wherein the residue R¹ of the hydroxy compound (I) is a branched or unbranched residue with or without heteroatom substituents and containing 3 to 9 carbon atoms.

3. A compound as claimed in claim 1, wherein the hydroxy compound (I) is at least one compound from the group 1-butanol, 2-butanol, isobutanol, 1-propanol, isopropanol, isononanol, 2-ethylhexan-1-ol and their alkoxylation products.

4. A compound as claimed in claim 1, wherein the epoxide of formula (III) is at least one compound from the group consisting of
ethylene glycol diglycidyl ether, 1,2-propanediol diglycidyl ether, 1,3-propanediol diglycidyl ether, 1,3-butanediol diglycidyl ether, 1,4-butanediol diglycidyl ether, 1,6-hexanediol diglycidyl ether, neopentyl glycol diglycidyl ether, cyclohexanedimethanol diglycidyl ether, diethylene glycol diglycidyl ether, dipropylene glycol diglycidyl ether, polyethylene glycol diglycidyl ether, polypropylene glycol diglycidyl ether, poly(ethylene-stat./block-propylene glycol) diglycidyl ether, resorcinol diglycidyl ether, 2,2-bis[4-(glycidyloxy)phenyl]propane, bis(4-glycidyloxyphenyl)methane, and bisphenol A propoxylate (1-PO/phenol)diglycidyl ether, diglycidyl 1,2-cyclohexanedicarboxylate, glycidyl glutarate, and glycidyl adipate.

5. A compound as claimed in claim 1, wherein the epoxide of formula (V) is at least one compound from the group trimethylolpropane triglycidyl ether, triphenylolmethane triglycidyl ether.

6. An ether alcohol of the general formula (VI) or (VII) 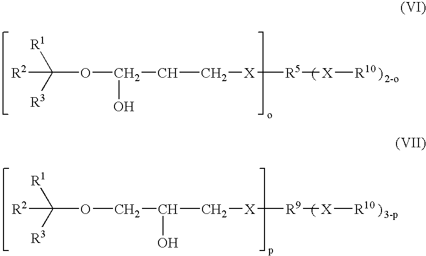
in which
X is an oxygen or a carboxyl group,
R¹ is a branched or unbranched, aromatic or nonaromatic, saturated or unsaturated residue with or without heteroatom substituents and containing 1 to 9 carbon atoms or can be R^{1a}, with the meaning as follows:
R^{1a} is oxyalkylene residue of the formula (III) 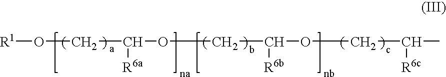
where
R^{6a},R^{6b} and R^{6c} independently of one another are hydrogen, methyl, ethyl, or another, branched or unbranched, unsaturated or saturated residue with or without heteroatom substituents and containing 1 to 10 carbon atoms, correspond optionally to aromatic radical having 6 to 8 carbon atoms, and
a, b, and c independently of one another are numbers between 0 and 3, and
na and nb independently of one another are numbers between o and 25, with 1<na+nb<25, with the proviso that both a random and a blockwise arrangement of the oxyalkylene units may be present,
R² and R³ independently of one another can be hydrogen or one of the residues R¹,
R⁵ is either a branched or unbranched, saturated or unsaturated, aromatic or nonaromatic residue with or without heteroatom substituents and containing 1 to 30 carbon atoms,
or R⁵ is an oxyalkylene residue R^{1b} of the formula (IIIb) 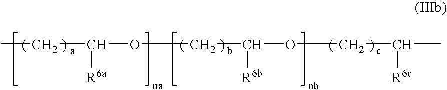
in which a, b, c, na, nb, R^{6a}, R^{6b} and R^{6c} are as defined above or
R⁵ is a residue of the formula (IV) 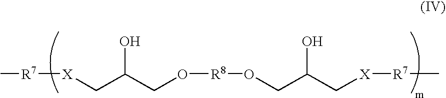
where
R⁷ is a branched or unbranched, saturated or unsaturated, aromatic or nonaromatic residue with or without heteroatom substituents and containing 1 to 30 carbon atoms, or is an oxyalkylene residue R^{1b} of the formula (IIIb), and
R⁸ is a branched or unbranched, saturated or unsaturated, aromatic or nonaromatic residue with or without heteroatom substituents and containing 1 to 30 carbon atoms, or likewise is an oxyalkylene residue R^{1b} of the formula (IIIb), and
R⁹ can be a branched or unbranched, saturated or unsaturated residue with or without heteroatom substituents or the residue R^{1b} of the formula (IIIb), in which the residues R^{6a}, R^{6b} and R^{6c} independently of one another are as defined above but at least once are branched or unbranched, saturated or unsaturated alkylene residues with or without heteroatom substituents and containing 1 to 10 carbon atoms,
R¹⁰ residues are any desired residues from the group of branched or unbranched, saturated or unsaturated residues with or without heteroatom substituents,
o is 1 to 2, and
p is 2 to 3.

7. An ether alcohol as claimed in claim 1, prepared by reacting neopentyl glycol diglycidyl ether and/or trimethylolpropane triglycidyl ether with 2-butanol or by reacting with at least one alcohol of the general formula (I), in which R¹ is at least one residue from the group of isobutanol, 1-butanol, 2-butanol, 1-propanol, isopropanol, isononanol, 2-ethylhexan-1-ol and their alkoxylation products in a substantially equimolar ratio of hydroxyl to epoxide groups.

8. An ether alcohol as claimed in claim 1, prepared by reacting at least one diglycidyl compound from the group of polypropylene glycol diglycidyl ether, polyethylene glycol diglycidyl ether, poly(ethylene-stat./block-propylene glycol) diglycidyl ether with at least one alcohol from the group of isobutanol, 1-butanol, 2-butanol, 1-propanol, isopropanol, isononanol, 2-ethylhexan-l-ol and their alkoxylation products in a substantially equimolar ratio of hydroxyl to epoxide groups.

9. An ether alcohol as claimed in claim 1, prepared by reacting polypropylene glycol diglycidyl ether with 2-butanol and/or isobutanol in a substantially equimolar ratio of hydroxyl to epoxide groups.

10. A method of reducing surface tension and inhibiting the (re)formation of foam in an aqueous formulation which comprises of adding the ether alcohol of claim 1 to the aqueous formulation.

11. The method of claim 10 wherein the reducing of surface tension and inhibiting of the (re)formation of foam occurs in a process for producing a surface coating, paint, printing ink or varnish.

12. An aqueous formulation comprising at least one ether alcohol of claim 1.

13. A compound as claimed in claim 3, wherein the epoxide of formula (III) is at least one compound from the group consisting of
ethylene glycol diglycidyl ether, 1,2-propanediol diglycidyl ether, 1,3-propanediol diglycidyl ether, 1,3-butanediol diglycidyl ether, 1,4-butanediol diglycidyl ether, 1,6-hexanediol diglycidyl ether, neopentyl glycol diglycidyl ether, cyclohexanedimethanol diglycidyl ether, diethylene glycol diglycidyl ether, dipropylene glycol diglycidyl ether, polyethylene glycol diglycidyl ether, polypropylene glycol diglycidyl ether, poly(ethylene-stat./block-propylene glycol)diglycidyl ether, resorcinol diglycidyl ether, 2,2-bis[4-(glycidyloxy)phenyl]propane, bis(4-glycidyloxyphenyl)methane, and bisphenol A propoxylate (1-PO/phenol)diglycidyl ether, diglycidyl 1,2-cyclohexanedicarboxylate, glycidyl glutarate, and glycidyl adipate.

14. A compound as claimed in claim 3, wherein the epoxide of formula (V) is at least one compound from the group trimethylolpropane triglycidyl ether, triphenylolmethane triglycidyl ether.
